(19) United States
(12) Patent Application Publication
(10) Pub. No.: US 2018/0064635 A1
(43) Pub. Date: Mar. 8, 2018

(54) COSMETIC COMPOSITION COMPRISING SLIME OF SNAIL FED WITH BERRY FRUITS AND PREPARATION METHOD THEREOF

(71) Applicants: CREALAND CO., LTD, Seongnam-si (KR); MEGACOS CO., LTD, Anyang-Si (KR)

(72) Inventors: Seok Kee Min, Seoul (KR); Jun Pil Min, Seoul (KR); Suk Kyun Jung, Seoul (KR); Byeong Chan Kang, Gyeonggi-do (KR); Sung Yong Park, Anyang-si (KR)

(21) Appl. No.: 15/557,055

(22) PCT Filed: Mar. 30, 2016
(86) PCT No.: PCT/KR2016/003284

§ 371 Date: Sep. 8, 2017

(30) Foreign Application Priority Data
Mar. 31, 2015 (KR) 10-2015-0045117

(51) Int. Cl. A61K 8/98; A61K 8/34; A61Q 19/08

(52) U.S. Cl. CPC A61K 8/987; A61K 8/347; A61K 2800/782; A61K 2800/522; A61Q 19/08

(57) ABSTRACT

The present invention relates to a cosmetic composition comprising slime of snails fed with berry fruits and a preparation method thereof. In particular, the present invention relates to a composition comprising slime of snails fed with berry fruits and a preparation method thereof, wherein snail slime obtained from snails fed with berry fruits enhances skin regeneration, skin elasticity, and moisture by means of mucin, collagen, and elastin contained in the snail slime such that an active oxygen in skin can be decreased and skin aging can be prevented by polyphenol contained in the berry fruits, and the slime of snails fed with berry fruits in the cosmetic composition of the present invention is harmless to a human body as a natural raw material and prevents skin problems by reducing skin stress caused by external environments.

### CROSS-REFERENCE TO RELATED APPLICATION

This application claims the benefit of Korean application No. 10-2015-0045117, filed on Mar. 31, 2015 with the Korean Intellectual Property Office, the disclosure of which is incorporated herein by reference.

### TECHNICAL FIELD

The present invention relates to a cosmetic composition comprising slime of snails fed with berry fruits and a preparation method thereof. In particular, the present invention relates to a composition comprising slime of snails fed with berry fruits and a preparation method thereof, wherein snail slime obtained from snails fed with berry fruits enhances skin regeneration, skin elasticity, and moisture by means of mucin, collagen, and elastin contained in the snail slime such that an active oxygen in skin can be decreased and skin aging can be prevented by polyphenol contained in the berry fruits, and the slime of snails fed with berry fruits in the cosmetic composition of the present invention is harmless to a human body as a natural raw material and prevents skin problems by reducing skin stress caused by external environments.

### BACKGROUND ART

Various physical and chemical changes occur in a human skin in the process of aging, and the cause is mainly classified into intrinsic aging and photo aging, which have been researched actively. The skin aging may be caused by activation of free radicals due to ultraviolet rays, stresses, disease conditions, environmental factors, wounds, and age. As those states are intensified, an antioxidant defense network in vivo is destroyed, and cells and tissues are damaged, thereby stimulating the skin aging.

In other words, main constituents of the skin such as lipids, proteins, polysaccharides and nucleic acids are oxidized, so that skin cells and tissues are destroyed, and finally a skin aging phenomenon occurs.

Particularly, the protein oxidation causes the cleavage of collagen, hyaluronic acid, elastin, proteoglycan, fibronectin and so on which are connective tissues of the skin, thus the elasticity of the skin is reduced.

Therefore, when cell membranes are protected by scavenging free radicals generated during body metabolism or free radicals mediated by ultraviolet radiation, and cells are multiplied by regenerating already damaged cells through active metabolism, the skin may be quickly recovered and a healthy skin may be maintained.

With respect to the related art recently and usually used and having an effect on skin aging prevention, Korean Registered Patent Publication No. 10-1315325 discloses a composition for skin external application containing antioxidant components for improving skin wrinkle and skin whitening. The above composition for skin external application contains at least, two antioxidant ingredients selected from the group consisting of lipoic acid, tocopherol, coenzyme Q10, selenium and vitamin C, so that free radicals are scavenged or the activity of intradermal protective factors related with antioxidation is increased, thereby having excellent effects on improving the skin wrinkle and skin whitening.

The composition for external application for skin of the related art is a substance having an effect on scavenging the free radicals, and has been used for preventing wrinkles and other skin diseases by mixing the composition with cosmetics or medicines. However, the composition of the related art is expensive and oxidized because of low stability when the composition is mixed, so that it may be difficult to obtain a substantial effect.

In other words, in order to reduce skin irritation caused by various stress sources and reduce side effects caused by the use of cosmetics, it is urgently required to develop a substance having effects on the antioxidation and mitigating the skin irritation. In addition, it is also required for a method capable of manufacturing the substance by using an active ingredient of a natural product having less side effects on the human body.

Accordingly, in order to improve the skin aging and troubles caused by the free radicals, it is essential to develop the natural product excellent in improving antioxidant effects and having no side effect, to develop cosmetics containing the natural product, and to develop an optimal manufacturing method.

With respect to the related art to improve the stability of the formulation and use natural materials in order to solve the above-mentioned conventional problems, Korean Registered Patent Publication No. 10-1208012 discloses a cosmetic composition containing slime of snails fed with red ginseng. More particularly, the disclosure relates to a cosmetic composition containing snail slime derived from snails fed with red ginseng.

The above related art can reduce manufacturing costs by using inexpensive natural materials instead of expensive raw materials, and a cosmetic composition harmless to a human body can be manufactured because the natural materials are used.

However, according to the above related art, since red ginseng powder is used for feeding the snails, the snails avoid the feed because the red ginseng smells and tastes strong. When the red ginseng powder is forcibly fed, the snails suffer from stress and cannot ingest the feed, thereby reducing the snail slime.

In addition, the above snails absorb small amounts of the red ginseng, so small amount of saponin which is an ingredient of the red ginseng is detected in the snail slime. The small amount of the saponin cannot exhibit its function, so that it is not suitable for addition to the cosmetic composition.

### DISCLOSURE

### Technical Problem

To solve the above problems, an object of the present invention is to provide a cosmetic composition containing slime of a snail fed with berry fruits and a method of preparing the same, in which snail slime is obtained from the snails fed with the berry fruit in order to reduce active oxygen in a skin and prevent skin aging by using polyphenol contained in berry fruits, and skin regeneration, skin elasticity and moisturizing ability are reinforced by using mucin, collagen and elastin contained in the snail slime.

In addition, an object of the present invention is to provide a cosmetic composition containing slime of snail—fed with berry fruits and a method of preparing the same, in which the slime of snails fed with berry fruits in the cosmetic composition of the present invention is harmless to a human body as a natural raw material, and prevents skin troubles by relaxing the skin stress caused by external environments.

### Technical Solution

According to the present invention, the snails are bred on a berry fruit sapling at the temperature of 20° C. to 35° C. and the humidity of 60% to 80%. The berry fruit is selected from blueberry, aronia berry, and acai berry. The snail slime containing mucin, collagen and elastin ingredients is obtained from the snails, and the snail slime includes polyphenol which is an ingredient of the berry fruit.

In addition, according to the present invention, the snail slime containing ingredients of the berry fruit is filtered in a filtration device equipped with a porous filter to obtain a snail slime filtrate.

In addition, according to the present invention, the snail slime filtrate further includes a collagen decomposition inhibitor or an elastin decomposition inhibitor.

Further, the present invention includes: a step (S10) of breeding the snails on a berry fruit sapling at the temperature: of 20° C. to 35° C. and the humidity of 60% to 80%; a step (S20) of collecting snail slime for 30 minutes to 8 hours after washing the snails with purified water and putting the snails into a net; a step (S30) of obtaining snail slime filtrate by filtering the snail slime in a filtration device equipped with a porous filter to obtain a snail slime filtrate; and a step (S50) of preparing a cosmetic composition by mixing the snail slime filtrate prepared in step S30 with cosmetic materials.

After step (S30), the present invention further includes a step (S40) of adding a collagen decomposition inhibitor or an elastin decomposition inhibitor into the snail slime filtrate.

### Advantageous Effects

According to the cosmetic composition containing the slime of the snail fed with the berry fruits and the method of preparing the same of the present invention as described above, active oxygen in a skin can be reduced and skin aging can be prevented by polyphenol contained in berry fruits by obtaining snail slime from the snails fed with the berry fruits, and skin regeneration, skin elasticity and moisturizing ability can be reinforced by using mucin, collagen and elastin contained in the snail slime.

In addition, according to the cosmetic composition containing the slime of the snail fed with the berry fruits and the method of preparing the same of the present invention, the slime of snails fed with berry fruits in the cosmetic composition is harmless to a human body as a natural raw material, and can prevent skin troubles by relaxing the skin stress caused by external environments.

### DESCRIPTION OF DRAWINGS

FIG. 1 is a flowchart describing a method of preparing a cosmetic composition containing the slime of the snail fed with the berry fruits according to the present invention.

### BEST MODE

[Mode for Invention]

According to the present invention, as a result of repeated researches to solve the above technical problems, the inventors of the present invention have developed a method of preparing a cosmetic composition containing slime of snails fed with berry fruits, and have completed the present invention by confirming that the cosmetic composition containing the slime of the snail fed with the berry fruits by using the above method can reduce active oxygen in the skin, and prevent the skin aging by using polyphenol contained in the berry fruits, and skin regeneration, skin elasticity and moisturizing ability can be reinforced by using mucin, collagen and elastin contained in the snail slime.

Hereinafter, the present invention will be described in detail.

According to the present invention, the snails are bred on a berry fruit sapling in a range of the temperature of 20° C. to 35° C. and the humidity of 60% to 80%. The berry fruit is one selected from blueberry, aronia berry, and acai berry. The snail slime containing mucin, collagen and elastin ingredients is obtained from the snails, and the snail slime includes polyphenol which is an ingredient of the berry fruit.

The snails are bred on a berry fruit sapling in a range of the temperature of 20° C. to 35° C. and the humidity of 60% to 80%, so that the snails are allowed to ingest the berry fruits well and discharge the snail slime sufficiently.

There are problems in that the snails lose their activity and do not ingest the berry fruits when the breeding temperature is less than 20° C. and the breeding humidity is less than 60%, the snail slime dries up when the breeding temperature is higher than 35° C., and the berry fruits are affected to quickly wither away when the breeding humidity is higher than 80%.

The snail slime is obtained from snails fed with the berry fruits, and the snail slime is obtained from snails fed with the berry fruits has abundant mucin, collagen and elastin compared with the conventional snail slime, so that it can help to improve a healthy skin by improving an unstable skin barrier and creating a new skin layer, a clean skin can be obtained by deactivating foreign substances harmful to the skin, and the skin can be elastic by maintaining moisturizing ability on the skin to contribute to the maintenance of moist skin.

In addition, polyphenol which is an ingredient of berry fruits is also obtained, so that free radicals in the skin can be reduced and the skin aging can be prevent.

According to the present invention, the snail slime containing ingredients of the berry fruit is filtered in a filtration device equipped with a porous filter to obtain a snail slime filtrate.

Foreign matters remaining when the snail slime is obtained with the porous filter are removed by using an ultra filtration (UF) hollow filter membrane, so that the filtration device obtains the snail slime filtrate having high purity.

The porous filter has about 100 billion fine pores, each having sizes of 30 to 500 nanometers (nm), and removes pollutants present in the snail slime.

Accordingly, the snail slime filtrate can be obtained by selective filtration since impurities are removed but beneficial substances in the snail slime are not filtered.

The snail slime obtained from snails fed with the berry fruits according to the present invention is contained in an amount of 0.001% to 30% by weight based on the total weight of the cosmetic composition, and preferably 0.1% to 15% by weight.

0.001% and 30% by weight of the above snail slime filtrate based on the total weight of the cosmetic composition are the minimum and maximum thresholds for achieving effects on skin anti-aging, skin regeneration, skin elasticity and moisturizing, respectively. When it is less than 0.001% by weight, the content of polyphenol which is the ingredient of the berry fruits and the content of mucin, collagen and elastin which are ingredients of the snail slime are too small, it rarely has the effects on skin regeneration, skin elasticity and moisturizing, and skin anti-aging. When it is more than 30% by weight, the effects on skin regeneration, skin elasticity and moisturizing ability and skin anti-aging are reduced to half.

In addition, 0.1% to 15% by weight of the snail slime filtrate based on the total weight of the cosmetic composition are the optimal thresholds for achieving the effects on skin aging prevention, skin regeneration, and skin elasticity and moisturizing, which are shown in Example 4.

According to the present invention, the snail slime filtrate further includes a collagen decomposition inhibitor or an elastin decomposition inhibitor.

The snail slime filtrate further includes the collagen decomposition inhibitor or the elastin decomposition inhibitor, thereby inhibiting the decomposition of the collagen and the elastin.

In addition, when the cosmetic material prepared with the snail slime filtrate containing the collagen decomposition inhibitor or the elastin decomposition inhibitor is applied onto the skin, the collagen decomposition inhibitor or the elastin decomposition inhibitor is absorbed in the skin, and the activity of matrix metalloproteinase (MMP) which is a collagenase and the activity of elastase which is an elastine decompositing enzyme are inhibited, thus destruction of the skin elastic fibers is prevented, and a skin damage caused by the exposure to ultraviolet rays is reduced, thereby helping to improve skin elasticity.

In addition, the collagen decomposition inhibitor or the elastin decomposition inhibitor contained in the snail slime filtrate acts together with polyphenol which is an ingredient of the berry fruits, thereby serving to maximize the effect on the skin anti-aging.

The collagen decomposition inhibitor is a tissue inhibitor of metalloproteinase (TIMP) protein, in which it is preferable that the TIMP is obtained by selecting at least one from the group consisting of retinol, green tea extract, phytoestrogen, vitamin E, and omega-3 fatty acids.

The elastin decomposition inhibitor is preferably a glycine soja protein.

Besides the snail slime obtained from the snails fed with the berry fruits, the cosmetic composition according to the present invention may contain other ingredients which are capable of improving the main effect without degrading the main effect of the present invention.

The composition according to the present invention may contain adjuvants commonly used in a cosmetic field such as hydrophilic or lipophilic gelling agents, hydrophilic or lipophilic active agents, preservatives, antioxidants, solvents, fragrance, fillers, blocking agents, pigments, absorbers and dyes. The amounts of the above various adjuvants are those commonly used in the art, for example, 0.001% to 30% by weight based on the total weight of the composition. However, in any case, the adjuvants and proportions thereof may be selected so as not to adversely affect the preferable properties of the cosmetic composition according to the invention.

In addition, it is preferable that the formulation of the cosmetic composition according to the present invention is selected from the base cosmetic formulation including toner, gel, water-soluble liquid, cream, essence, oil-in-water (O/W) or water-in-oil (W/O) type emulsion, and ointment, and the color cosmetic formulation including O/W or W/O type makeup base, foundation, skin cover, lipstick, lip gloss, face powder, two-way cake, eyeshadow, cheek color and eyebrow pencil.

Further, the present invention may be applied to the skin in the form of an aerosol, or in the form a solid such as a stick, and may be used for a skin care product and/or a makeup product.

The method of preparing the cosmetic composition containing the slime of the snail fed with the berry fruits according to the present invention will be described as follows with reference to the accompanying drawings.

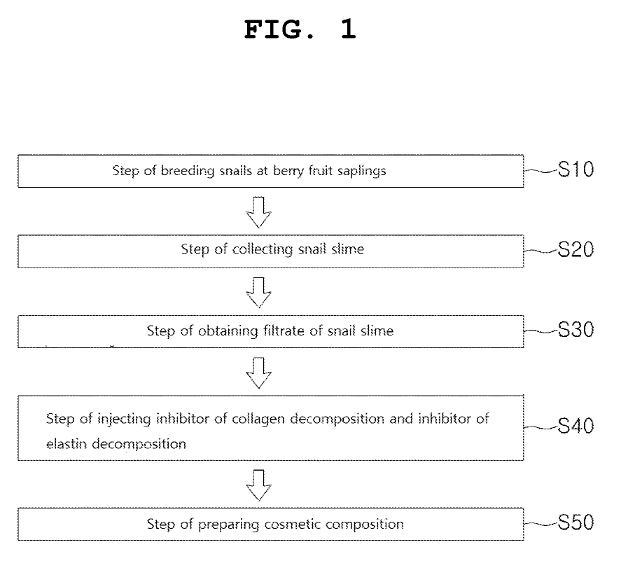

FIG. 1 is a flowchart describing a method of preparing a cosmetic composition containing the slime of the snail fed with the berry fruits according to the present invention.

Hereinafter, preferred embodiments of the present invention will be described with reference to the accompanying drawings. The method according to the present invention preferably includes the following steps, but it is not limited thereto.

The present invention includes: a step (S10) of breeding the snails on a berry fruit sapling in a range of the temperature of 20° C. to 35° C. and the humidity of 60% to 80%; a step (S20) of collecting snail slime for 30 minutes to 8 hours after washing the snails with purified water and putting the snails into a net; a step (S30) of obtaining snail slime filtrate by filtering the snail slime in a filtration device equipped with a porous filter to obtain a snail slime filtrate; and a step (S50) of preparing a cosmetic composition by mixing the snail slime filtrate prepared in step S30 with cosmetic materials.

In addition, after step S30, the present invention further includes a step (S40) of injecting a collagen decomposition inhibitor or an elastin decomposition inhibitor into the snail slime filtrate.

The manufacturing method of the present invention will be described separately for each step as follows.

Step S10 of breeding the snails on a berry fruit sapling in a range of the temperature of 20° C. to 35° C. and the humidity of 60% to 80% is performed.

The above temperature and humidity are optimal states for enabling the snails to be active. When the temperature and humidity are out of the above range, the snails are not active properly and do not ingest fruits such as blueberry, aronia berry, and acai berry, thereby reducing the contents of polyphenol which is an ingredient of the blueberry, aronia berry and acai berry in the snail slime.

In addition, because the snails are not compulsively fed with the feed, but bred and pastured freely in saplings, the snails can produce snail slime containing the polyphenol without stress.

Step S20 of collecting snail slime for 30 minutes to 8 hours after washing the snails with purified water and putting the snails into a net is performed.

Snails try to cover and protect their bodies by discharging the slime when the snails are in a dangerous situation or a bad environment, so the snails sense the danger and start to discharge the slime when it takes 30 minutes after the snails are put into a net in large quantities.

Accordingly, when a collecting time is less than 30 minutes, the snail slime can not be obtained, and when the collecting time exceeds eight hours, the snails excessively suffer from stress to die.

Step S30 of obtaining snail slime filtrate by filtering the snail slime in a filtration device equipped with a porous filter to obtain a snail slime filtrate is performed.

The filtration device is an ultra filtration (UF) hollow filter membrane equipped with a porous filter and having porosities with sizes of 30 to 500 nanometers (nm), and obtains snail slime filtrate having high purity by removing foreign matters remaining upon obtaining the snail slime using the filtration device.

Step S50 of preparing a cosmetic composition by mixing the snail slime filtrate prepared in step S30 with cosmetic materials is performed.

Cosmetics for enhancing skin anti-aging, skin regeneration, and skin elasticity and moisturizing ability may be manufactured by preparing a cosmetic composition using the snail slime filtrate.

After step S30, step S40 of further injecting a collagen decomposition inhibitor or an elastin decomposition inhibitor into the snail slime filtrate is performed.

The decompositions of the collagen and the elastin in the snail slime filtrate and the skin are inhibited by inputting the collagen decomposition inhibitor or the elastin decomposition inhibitor.

In addition, the collagen decomposition inhibitor or the elastin decomposition inhibitor contained in the snail slime filtrate acts together with polyphenol which is an ingredient of the berry fruits, thereby serving to maximize the effect on the skin anti-aging.

The collagen decomposition inhibitor is a tissue inhibitor of metalloproteinase (TIMP) protein, in which it is preferable that the TIMP is obtained by selecting at least one from the group consisting of retinol, green tea extract, phytoestrogen, vitamin E, and omega-3 fatty acids.

Preferably, the elastin decomposition inhibitor is a glycine soja protein.

The present invention will be described in detail with reference to examples, comparative examples, experimental examples, and formulation examples.

However, the following examples, comparative examples, experimental examples and formulation examples are merely illustrative of the present invention, the present invention is not limited to the following examples, comparative examples, experimental examples and formulation Examples.

### EXAMPLE 1

### Example 1 of Preparation of Snail Slime Filtrate from Snails Fed with Berry Fruits

Example 1 of preparation of snail slime filtrate from snails fed with berry fruits according to the present invention was prepared through the following preparation step.

(S10): The snails were bred on a berry fruit sapling in a range of the temperature of 25° C. and the humidity of 65%.

(S20): Snail slime was collected for 5 hours after the snails were washed with purified water and put into a net.

(S30): the snail slime filtrate was obtained by filtering the snail slime through a filtration device equipped with a porous filter having porosities with sizes of 250 nanometers (nm), and was stored in a low-temperature refrigerator maintained at 5° C. to prevent the snail slime filtrate from being denatured.

### EXAMPLE 2

### Example 2 of Preparation of Snail Slime Filtrate from Snails Fed with Berry Fruits

Example 2 of preparation of snail slime filtrate from snails fed with berry fruits according to the present invention was prepared through the following preparation steps.

(S10): The snails were bred on a berry fruit sapling in a range of the temperature of 25° C. and the humidity of 65%.

(S20): Snail slime was collected for 5 hours after the snails were washed with purified water and put into a net.

(S30): The snail slime filtrate was obtained by filtering the snail slime through a filtration device equipped with a porous filter having porosities with sizes of 250 nanometers (nm).

(S40): After injecting a collagen decomposition inhibitor or an elastin decomposition inhibitor into the snail slime filtrate, the snail slime filtrate finished in preparation was stored in a low-temperature refrigerator maintained at 5° C.

### COMPARATIVE EXAMPLE 1

### Comparative Example 1 of Preparation of Snail Slime Filtrate from Snails Not Fed with Berry Fruits

Comparative Example 1 of preparation of snail slime filtrate from snails not fed with berry fruits is prepared through the following production steps.

(S10): Snails were bred in a range of the temperature of 25° C. and the humidity of 65%.

(S20): Snail slime was collected for 5 hours after the snails were washed with purified water and put into a net.

(S30): The snail slime filtrate was obtained by filtering the snail slime through a filtration device equipped with a porous filter having porosities with sizes of 250 nanometers (nm), and was stored in a low-temperature refrigerator maintained at 5° C. to prevent the snail slime filtrate from being denatured.

### COMPARATIVE EXAMPLE 2

### Comparative Example 2 of Preparation of Snail Slime Filtrate from Snails Fed with Red Ginseng

Comparative Example 2 of preparation of snail slime filtrate from snails fed with red ginseng is prepared through the following production steps.

(S10): The snails were bred while being fed with red ginseng powder in a range of the temperature of 25° C. and the humidity of 65%.

(S20): Snail slime was collected for 5 hours after the snails were washed with purified water and put into a net.

(S30): The snail slime filtrate was obtained by filtering the snail slime through a filtration device equipped with a porous filter having porosities with sized of 250 nanometers (nm), and was stored in a low-temperature refrigerator maintained at 5° C. to prevent the snail slime filtrate from being denatured.

### EXPERIMENTAL EXAMPLE 1

### Measurement of Antioxidant Effect of Snail Slime Filtrates Prepared in Examples 1 and 2 and Comparative Examples 1 and 2

A free radical scavenging test and a reactive oxygen scavenging test were performed in order to confirm the antioxidant effect of the snail slime filtrates prepared in Examples 1 and 2 and Comparative Examples 1 and 2, as a verifying test for a skin aging improvement effect.

The free radical scavenging test was carried out through a verifying test scheme under the conditions that the absorbance of stable DPPH exhibits the maximum absorbance at 540 nm, and the absorbance at the above wavelength of 540 nm is reduced as the free radical scavenging rate is increased, that is, as the free radical DPPH is scavenged by the sample and becomes purple to transparent.

The snail slime filtrates prepared in the above Examples 1 and 2 and Comparative Examples 1 and 2 were diluted with methanol in a predetermined concentration, and 1 ml of the dilution was mixed with 1 ml of a 0.1 mM 2,2-diphenyl-1-picryl-hydrazyl radical (DPPH, Sigma) solution. After the mixture leaves at 37° C. for 15 minutes, the absorbance was measured at the wavelength of 540 nm by using a microplate reader (Thermo max, USA).

In the above free radical scavenging test, the control group was measured In the same manner by inputting 1 ml of DPPH and 1 ml of methanol, and color correction values were obtained with respect to each of the sample and the control group by inputting 1 ml of methanol and 1 ml of sample.

SC₅₀ is the concentration of the sample required to scavenge 50% of the free radicals, and the smaller value indicates the higher antioxidant activity.

In the active oxygen scavenging test, changes on the absorbance are measured using the oxidation of nitroblue tetrazolium (NBT) by active oxygen by using active oxygen generation upon enzymatic reaction of xanthine/xanthine oxidase (Sigma), thus the scavenging ability of the active oxygen can be confirmed.

2.4 ml of sodium carbonate (Na2CO3), 0.1 ml of xanthine (Sigma), 0.1 ml of ethylene diamine tetra acetic acid (EDTA), 0.1 ml of bovine serum albumin (BSA, Sigma), 0.1 ml of NBT, and 1 ml of the snail slime filtrates prepared in Examples 1 and 2 and Comparative Examples 1 and 2 were added, vortexing was carried out by using a vortex mixer (Type 37600 Mixer, Mini mix, USA), the mixture was left at 25° C. for 10 minutes. After 0.1 ml of xanthine oxidase was added and reacted at 25° C. for 20 minutes, the reaction was stopped by inputting 6 mM of CuCl2, and the absorbance was measured at the wavelength of 540 nm by using a microplate reader (UVT-06685, Thermo max, USA).

The control group in the active oxygen scavenging activity test was measured in the same manner by inputting third distilled water instead of the sample solution. Third distilled water was inputted instead of xanthine oxidase solution to obtain color correction values with respect to the extracted sample and the control group.

IS₅₀ is the concentration of the sample required to scavenge 50% of the active oxygen, and the smaller value indicates the higher antioxidant activity.

As a result, as shown in Table 1, the snail slime filtrates prepared in Examples 1 and 2 showed antioxidant abilities almost similar to that of BHT which is a strong synthetic antioxidant. The concentrations of 50% of the free radical scavenging ratio of the snail slime filtrates prepared in Examples 1 and 2 were 134 ppm and 110 ppm, respectively, and the concentrations of 50% of the active oxygen scavenging ratio of the snail slime filtrate prepared in Examples 1 and 2 were 162 ppm and 136 ppm, respectively, which indicates that the antioxidant activity was excellent at a small amount of the concentration.

In addition, it was found that, in the snail slime filtrates prepared in Examples 1 and 2, the polyphenol, which is an ingredient of a blueberry was sufficiently discharged into the snail slime enough to exert the antioxidant effect. It was confirmed that mucin, collagen, and elastin which are ingredients of the snail slime were mixed, so that the antioxidant efficacy was superior to those of Comparative Examples 1 and 2.

<Equation 1>

Free radical scavenging rate (%)=100−((absorbance of sample/absorbance of control group)×100)

<Equation 2>

Active oxygen scavenging rate (%)=100−((absorbance of sample/absorbance of control group)×100)

TABLE 1
Results of measurement of antioxidant effect

| | Free radical | Active oxygen |
|---|---|---|
| | scavenging rate | scavenging rate |
| | SC₅₀ (ppm) | IC₅₀ (ppm) |
| BHT | 125 | 154 |
| Example 1 | 134 | 162 |
| Example 2 | 110 | 136 |
| Comparative | 378 | 448 |
| Example 1 | | |
| Comparative | 289 | 373 |
| Example 2 | | |

### EXPERIMENTAL EXAMPLE 2

### Measurement of Skin Improvement Effect of Snail Slime Filtrates Prepared in Examples 1 and 2 and Comparative Examples 1 and 2

A test on inhibition of elastin decomposition activity was performed to verify the skin improvement effect of the snail slime filtrates prepared in Examples 1 and 2 and Comparative Examples 1 and 2.

Elastin is a component of a matrix layer in a dermis, and the elastin is decomposed and the matrix layer in the dermis collapse as the skin ages, thus wrinkles generates.

A scheme for measuring the activity of the elastase was used by measuring the absorbance was measured at a wavelength of 405 nm with respect to changes in color caused by the decomposition of p-nitroaniline, by using N-succinyl-(Ala)3 p-nitroaniline (Sigma) which is an elastase matrix, so as to measure the activity of elastase which is an enzyme for decomposing the elastine based on the above principle.

The control group for measuring the activity of the elastase was measured in the same manner by inputting the third distilled water instead of the sample, and the third distilled water was inputted instead of an enzyme solution to obtain a color correction value for each. Regarding a sample, phenyl methyl sulfonyl fluoride (PMSF, Sigma) as an elastase-specific inhibitor was used as a comparative group so as to determine an inhibiting effect on the activity of the elastase with respect to the snail slime filtrates prepared in Examples 1 and 2 and Comparative Examples 1 and 2. The elastase inhibition rate was calculated as numerical values by using the following equation 3.

IC₅₀ is the concentration of the sample required to inhibit the elastase activity by 50%, and the smaller value indicates the inhibition rate is higher.

Trizma-HCl (Sigma) of pH 8.0 and 0.267 M was used as a buffer solution, N-Succinyl-(Ala) 3 p-nitroaniline of 8.8 mM was used as a matrix solution, and an enzyme solution including porcine pancreatic elassthese having the concentration of 10 μg/ml (Sigma) was used. After 60 μl of the buffer solution, 20 μl of the matrix solution and the snail slime filtrates prepared in Examples 1 and 2 and Comparative Examples 1 and 2 were mixed with 100 μl of the sample solution diluted with the third distilled water for each concentration, respectively, 20 μl of the enzyme solution was inputted and reacted in a constant temperature water bath at 25° C. for 15 minutes to measure the generating amount of p-nitroaniline at a wavelength of 405 nm by using a microplate reader (UVT-06685, Thermo max, USA).

As a result, as shown in Table 2, the elastase activity inhibition rate of the snail slime filtrate prepared in Example 2 was 1.10 mg/ml, which was lower than 0.07 mg/ml of the elastase-specific inhibitor (PMSF), however, it was more effective in the skin improvement than retinol which is currently effective as a skin improving functional material.

In addition, it was confirmed that the skin improvement effect was superior to those of Comparative Examples 1 and 2 since the snail slime filtrates prepared in Examples 1 and 2 contain a collagen decomposition inhibitor or an elastin decomposition inhibitor, the skin elastic fibers are prevented from being destructed.

<Equation 3>

Elastase activity inhibition rate (%)=100−((absorbance of sample/absorbance of control group)×100)

TABLE 2
Results of measurement of skin improving effect

| | Elastase activity inhibition rate |
|---|---|
| | IC₅₀ (ppm) |
| Retinol | 1.20 |
| PMSF | 0.07 |
| Example 1 | 1.25 |
| Example 2 | 1.10 |
| Comparative Example 1 | 2.13 |
| Comparative Example 2 | 1.89 |

### EXPERIMENTAL EXAMPLE 3

### Measurement of Moisturizing Effect of Snail Slime Filtrates Prepared in Examples 1 and 2 and Comparative Examples 1 and 2

Measurement of moisturizing effect of snail slime filtrates prepared in Examples 1 and 2 and Comparative Examples 1 and 2 was carried out by a capacitance measurement scheme using an oil and moisture analyzer (WSK-P500U, Inforward. Inc., Japan). 1 wt % an aqueous solution of the snail slime filtrates prepared in Examples 1 and 2 and Comparative Examples 1 and 2 was compared with 1 wt % aqueous solution of hyaluronic acid sodium salt (Sigma) which is known to be excellent in a skin moisturizing effect based on various studies.

30 females in their 20s and 30s were targeted, a flexor surface of the forearm was allowed to acclimate to a constant temperature and humidity chamber (22° C. and 40-60% of relative humidity) for 20 minutes right before the test starts, and the moisture of the skin was constantly maintained. Then, after measuring the initial moisture content of an application site of each sample using the oil and moisture analyzer (WSK-P500U, Inforward. Inc., Japan), each of the samples at 2.0 mg/cm2 was applied at a different position for each examinee to minimize an error according to the flexor surface of the forearm. The skin moisture was firstly measured at time intervals of 10 minutes, 30 minutes, 50 minutes, 90 minutes, and 120 minutes after the application, and after 2 hours, the skin moisture was measured again.

Each measurement site was measured repeatedly for three times and skin moisture was calculated by the average value.

As a result, as shown in Table 3, it was confirmed that the moisturizing effect of 1 wt % aqueous solution of snail slime filtrates prepared in Examples 1 and 2 was superior when compared with 1 wt % aqueous solution of hyaluronic acid which is currently known to have an excellent moisturizing effect.

In addition, it was found that the snails (Examples 1 and 2) fed with the blueberry discharge snail slime which is more useful in skin moisturizing than snail slime from normal snails (Comparative Example 1) and snail slime from snails (Comparative Example 2) fed with red ginseng.

TABLE 3
Results of measurement of moisturizing effect

| | Moisture content (A.U) | | | | |
|---|---|---|---|---|---|
| | subject to time after application | | | | |
| | 10 | 30 | 50 | 90 | 120 |
| | minutes | minutes | minutes | minutes | minutes |
| 1 wt % of hyaluronic | 61.31 | 58.24 | 57.21 | 55.87 | 52.11 |
| acid | | | | | |
| 1 wt % Example 1 | 71.56 | 70.23 | 69.72 | 67.35 | 65.11 |
| 1 wt % Example 2 | 79.88 | 78.12 | 76.98 | 74.33 | 72.15 |
| 1 wt % Comparative | 45.72 | 43.98 | 41.32 | 40.08 | 38.78 |
| Example 1 | | | | | |
| 1 wt % Comparative | 53.33 | 51.22 | 50.08 | 48.76 | 44.38 |
| Example 2 | | | | | |

### EXPERIMENTAL EXAMPLE 4

### Measurement of Effects on Antioxidation, Skin Improvement, and Skin Moisturizing According to Content of Snail Slime Filtrate

According to Example 3 of the cosmetic composition containing snail slime from snails fed with berry fruits of the present invention, the cosmetic composition containing snail slime from snails fed with berry fruits was prepared by containing 0.0005% by weight of the snail slime filtrate prepared in Example 2 based on the total weight of the cosmetic composition.

According to Example 4 of the cosmetic composition containing snail slime from snails fed with berry fruits of the present invention, the cosmetic composition containing snail slime from snails fed with berry fruits was prepared by containing 0.001% by weight of the snail slime filtrate prepared in Example 2 based on the total weight of the cosmetic composition.

According to Example 5 of the cosmetic composition containing snail slime from snails fed with berry fruits of the present invention, the cosmetic composition containing snail slime from snails fed with berry fruits was prepared by containing 0.1% by weight of the snail slime filtrate prepared in Example 2 based on the total weight of the cosmetic composition.

According to Example 6 of the cosmetic composition containing snail slime from snails fed with berry fruits of the present invention, the cosmetic composition containing snail slime from snails fed with berry fruits was prepared by containing 15% by weight of the snail slime filtrate prepared in Example 2 based on the total weight of the cosmetic composition.

According to Example 7 of the cosmetic composition containing snail slime from snails fed with berry fruits of the present invention, the cosmetic composition containing snail slime from snails fed with berry fruits was prepared by containing 30% by weight of the snail slime filtrate prepared in Example 2 based on the total weight of the cosmetic composition.

According to Example 8 of the cosmetic composition containing snail slime from snails fed with berry fruits of the present invention, the cosmetic composition containing snail slime from snails fed with berry fruits was prepared by containing 40% by weight of the snail slime filtrate prepared in Example 2 based on the total weight of the cosmetic composition.

The measurement of effects on antioxidation, skin improvement, and skin moisturizing according to content of snail slime filtrate is evaluated by the schemes of Test Examples 1 to 3, and the results are shown in Table 4. (The skin moisture content was measured in 120 minutes after applying the cosmetic composition onto the skin.)

TABLE 4
Results of measurement of antioxidation, skin improvement, skin moisturizing
effects according to contents of snail slime filtrate

| | Example 3 | Example 4 | Example 5 | Example 6 | Example 7 | Example 8 |
|---|---|---|---|---|---|---|
| | 0.0005 wt % | 0.001 wt % | 0.1 wt % | 15 wt % | 30 wt % | 40 wt % |
| Free radical | X | 142 | 131 | 118 | 128 | 177 |
| scavenging rate | | | | | | |
| SC₅₀ (ppm) | | | | | | |
| Active oxygen | X | 178 | 145 | 138 | 160 | 234 |
| scavenging rate | | | | | | |
| IC₅₀ (ppm) | | | | | | |
| Elastase activity | X | 1.68 | 1.23 | 1.18 | 1.39 | 1.73 |
| inhibition rate | | | | | | |
| IC₅₀ (ppm) | | | | | | |
| Skin moisture | 39.71 | 48.14 | 51.56 | 71.38 | 68.78 | 65.14 |
| (AU) | | | | | | |

As shown in Table 4, when the content of snail slime filtrate is less than 0.001 wt %, the effects on skin regeneration, skin elasticity and moisturizing, and skin anti-aging are rarely exist, and 0.001 wt % and 30 wt % of the snail slime filtrate based on the total weight of the cosmetic composition were the minimum and maximum thresholds for achieving the effects on skin anti-aging, skin regeneration, skin elasticity and moisturizing, respectively.

In addition, 0.1 wt % to 15 wt % of the snail slime filtrate based on the total weight of the cosmetic composition are the optimal thresholds for achieving the effects on the skin anti-aging, the skin regeneration, the skin elasticity and moisturizing.

### FORMULATION EXAMPLE

### Components of the Cosmetic Composition Containing Snail Slime Filtrate Prepared in Example 2

Nutrition cream, softening toner, and nutrition toner are prepared by using the snail slime filtrate prepared in Example 2. The cosmetics are merely illustrative of the present invention and the present invention is not limited thereto.

TABLE 5
Nutrition cream

| | | Contents |
|---|---|---|
| | Ingredients | (wt %) |
| 1 | lipophilic glyceryl monosterate | 2.0 |
| 2 | stearic acid | 1.5 |
| 3 | cetearyl alcohol | 2.2 |
| 4 | beeswax | 1.0 |
| 5 | squalane | 3.0 |
| 6 | phyto-hydrogenated oil | 1.0 |
| 7 | sorbitan stearate | 0.6 |
| 8 | mineral oil | 5.0 |
| 9 | polysorbate 60 | 1.5 |
| 10 | dimethicone | 1.0 |
| 11 | trioctanoin | 5.0 |
| 12 | betaine | 3.0 |
| 13 | triethanolamine | 1.0 |
| 14 | glycerin | 5 0 |
| 15 | sodium hyaluronate | 3.0 |
| 16 | snail slime filtrate of Example 2 | 5.0 |
| 17 | distilled water | remaining |
| 18 | preservative, fragrance, pigment | small amount |

TABLE 6
Softening toner

| | | Contents |
|---|---|---|
| | Ingredients | (wt %) |
| 1 | glycerin | 5.00 |
| 2 | 1,3-butylene glycol | 3.00 |
| 3 | PEG 1500 | 1.00 |
| 4 | allantoin | 0.10 |
| 5 | DL-panthenol | 0.30 |
| 6 | EDTA-2Na | 0.02 |
| 7 | benzophenone-9 | 0.04 |
| 8 | ethanol | 10.00 |
| 9 | octyldodeceth-16 | 0.20 |
| 10 | polysorbate 20 | 0.20 |
| 11 | snail slime filtrate of Example 2 | 13.00 |
| 12 | preservative, fragrance, pigment | small amount |
| 13 | distilled water | remaining |

TABLE 7
Nutrition toner

| | | Contents |
|---|---|---|
| | Ingredients | (wt %) |
| 1 | cetearyl alcohol | 1.00 |
| 2 | glyceryl stearate | 0.50 |
| 3 | polysorbate 60 | 1.00 |
| 4 | sorbitan sesquioleate | 0.30 |
| 5 | cetyl octanoate | 6.00 |
| 6 | squalane | 4.00 |
| 7 | safflower oil | 4.00 |
| 8 | butylene glycol | 4.00 |
| 9 | glycerin | 4.00 |
| 10 | carbomer | 0.10 |
| 11 | triethanolamine | 0.10 |
| 12 | snail slime filtrate of Example 2 | 5.00 |
| 13 | preservative, fragrance, pigment | small amount |
| 14 | distilled water | remaining |

Herein, the description of the present invention is merely an embodiment of a cosmetic composition containing slime of the snail fed with berry fruits and a method for preparing the same. The present invention is not limited to the above embodiments, and may be embodied in various forms. The embodiments are provided to complete the disclosure of the present invention and clearly teach the scope of the invention to those having ordinary knowledge in the technical field of the present invention, and the present invention is only defined by the scope of the claims.

1. A cosmetic composition containing snail slime of snails fed with berry fruits, the cosmetic composition comprising the snail slime including mucin, collagen and an elastin ingredients obtained from the snails bred on a berry fruit sapling, wherein the snail slime includes polyphenol which is an ingredient of the berry fruits.

2. The cosmetic composition of claim 1, wherein the berry fruit is selected from blueberry, aronia berry, and acai berry.

3. The cosmetic composition of claim 1, wherein the snails are bred under a condition of a breeding temperature of 20° C. to 35° C. and a breeding humidity of 60% to 80%.

4. The cosmetic composition of claim 1, wherein the snail slime containing ingredients of the berry fruits is snail slime filtrate obtained by filtering the snail slime through a filtration device equipped with a porous filter having porosities with sizes of 30 to 500 nanometers (nm).

5. The cosmetic composition of claim 4, wherein the snail slime filtrate is contained in an amount of 0.001% to 30% by weights based on a total weight of the cosmetic composition.

6. The cosmetic composition of claim 4, wherein the snail slime filtrate comprises a collagen decomposition inhibitor or an elastin decomposition inhibitor.

7. The cosmetic composition of claim 6, wherein the collagen decomposition inhibitor is a tissue inhibitor of metalloproteinase (TIMP) protein, in which the TIMP includes one selected from the group consisting of retinol, green tea extract, phytoestrogen, vitamin E and omega-3 fatty acids.

8. The cosmetic composition of claim 6, wherein the elastin decomposition inhibitor is glycine soja protein.

9. The cosmetic composition of claim 8, wherein the cosmetic composition is used for skin anti-aging, skin regeneration, skin elasticity, and skin moisturizing.

10. The cosmetic composition of claim 8, wherein the cosmetic composition is selected from toner, gel, water-soluble liquid, cream, essence, oil-in-water (O/W) or water-in-oil (W/O) type emulsion, ointment, O/W or W/O type makeup base, foundation, skin cover, lipstick, lip gloss, face powder, two-way cake, eyeshadow, cheek color, and eyebrow pencil.

11. A method of preparing a cosmetic composition containing slime of snails fed with berry fruits, the method comprising:
breeding snails on a berry fruit sapling (S10);
collecting snail slime for 30 minutes to 8 hours after washing the snails with purified water and putting the snails into a net (S20);
obtaining snail slime filtrate by filtering the snail slime in a filtration device equipped with a porous filter having porosities with sizes of 30 to 500 nanometers (nm) (S30); and
preparing the cosmetic composition by mixing the snail slime filtrate prepared in step S30 with cosmetic materials (S50).

12. The method of claim 11, after step S30, further comprising injecting a collagen decomposition inhibitor or an elastin decomposition inhibitor into the snail slime filtrate (S40).

13. The method of claim 11, wherein the snails are bred at a breeding temperature of 20° C. to 35° C. and breeding humidity of 60% to 80% in step S10.

14. The cosmetic composition of claim 7, wherein the cosmetic composition is used for skin anti-aging, skin regeneration, skin elasticity, and skin moisturizing.

15. The cosmetic composition of claim 6, wherein the cosmetic composition is used for skin anti-aging, skin regeneration, skin elasticity, and skin moisturizing.

16. The cosmetic composition of claim 5, wherein the cosmetic composition is used for skin anti-aging, skin regeneration, skin elasticity, and skin moisturizing.

17. The cosmetic composition of claim 4, wherein the cosmetic composition is used for skin anti-aging, skin regeneration, skin elasticity, and skin moisturizing.

18. The cosmetic composition of claim 3, wherein the cosmetic composition is used for skin anti-aging, skin regeneration, skin elasticity, and skin moisturizing.

19. The cosmetic composition of claim 2, wherein the cosmetic composition is used for skin anti-aging, skin regeneration, skin elasticity, and skin moisturizing.

20. The cosmetic composition of claim 1, wherein the cosmetic composition is used for skin anti-aging, skin regeneration, skin elasticity, and skin moisturizing.

21. The cosmetic composition of claim 7, wherein the cosmetic composition is selected from toner, gel, water-soluble liquid, cream, essence, oil-in-water (O/W) or water-in-oil (W/O) type emulsion, ointment, O/W or W/O type makeup base, foundation, skin cover, lipstick, lip gloss, face powder, two-way cake, eyeshadow, cheek color, and eyebrow pencil.

22. The cosmetic composition of claim 6, wherein the cosmetic composition is selected from toner, gel, water-soluble liquid, cream, essence, oil-in-water (O/W) or water-in-oil (W/O) type emulsion, ointment, O/W or W/O type makeup base, foundation, skin cover, lipstick, lip gloss, face powder, two-way cake, eyeshadow, cheek color, and eyebrow pencil.

23. The cosmetic composition of claim 5, wherein the cosmetic composition is selected from toner, gel, water-soluble liquid, cream, essence, oil-in-water (O/W) or water-in-oil (W/O) type emulsion, ointment, O/W or W/O type makeup base, foundation, skin cover, lipstick, lip gloss, face powder, two-way cake, eyeshadow, cheek color, and eyebrow pencil.

24. The cosmetic composition of claim 4, wherein the cosmetic composition is selected from toner, gel, water-soluble liquid, cream, essence, oil-in-water (O/W) or water-in-oil (W/O) type emulsion, ointment, O/W or W/O type makeup base, foundation, skin cover, lipstick, lip gloss, face powder, two-way cake, eyeshadow, cheek color, and eyebrow pencil.

25. The cosmetic composition of claim 3, wherein the cosmetic composition is selected from toner, gel, water-soluble liquid, cream, essence, oil-in-water (O/W) or water-in-oil (W/O) type emulsion, ointment, O/W or W/O type makeup base, foundation, skin cover, lipstick, lip gloss, face powder, two-way cake, eyeshadow, cheek color, and eyebrow pencil.

26. The cosmetic composition of claim 2, wherein the cosmetic composition is selected from toner, gel, water-soluble liquid, cream, essence, oil-in-water (O/W) or water-in-oil (W/O) type emulsion, ointment, O/W or W/O type makeup base, foundation, skin cover, lipstick, lip gloss, face powder, two-way cake, eyeshadow, cheek color, and eyebrow pencil.

27. The cosmetic composition of claim 1, wherein the cosmetic composition is selected from toner, gel, water-soluble liquid, cream, essence, oil-in-water (O/W) or water-in-oil (W/O) type emulsion, ointment, O/W or W/O type makeup base, foundation, skin cover, lipstick, lip gloss, face powder, two-way cake, eyeshadow, cheek color, and eyebrow pencil.
